(19) United States
(12) Patent Application Publication
(10) Pub. No.: US 2006/0253148 A1
(43) Pub. Date: Nov. 9, 2006

(54) Apparatus and method of using an occluder for embolic protection

(76) Inventors: James Ernest Leone, Miami, FL (US); Jeffrey Wilson, Wrentham, MA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/121,879

(22) Filed: May 4, 2005

(51) Int. Cl. A61M 29/00

(52) U.S. Cl. 606/200

(57) ABSTRACT

An apparatus and method for occluding the flow of embolic blood from entering the brain or other arteries during surgical procedures. The apparatus includes an expandable coiled occluder. The occluder is deployed in a coiled state into a body lumen where it then at least partially uncoils and at least partially conforms to the diameter of the lumen. The method includes introducing an occluder into a body lumen; having the occluder in a deployed/uncoiled position when emboli are likely to be released; and, if determined to be necessary, having the occluder removed or coiled when emboli are not likely to be released. The occluder has an open center to permit the flow of blood through the lumen while restricting the flow of blood and hence embolic matter through or into side or branch vessels. An occluder having an open center disposed in the lumen of the aorta across the aortic arch vessels restricts the passage of embolic matter into the aortic arch vessels while allowing the flow of blood through the aorta to distal portions of the body during cardiac surgery.

### BACKGROUND

The present disclosure generally relates to surgical procedures requiring blood flow to bypass organs or major vessels during surgery. More specifically, the disclosure relates to an occluder apparatus and having the occluder apparatus in place to occlude the flow of blood at key times during surgery when emboli are likely to be dislodged.

### TECHNICAL FIELD

Over recent decades tremendous advances have been made in the area of heart surgery, including such life saving surgical procedures as coronary artery bypass grafting (CABG) and cardiac valve repair or replacement. Cardiopulmonary bypass is an important enabling technology that has helped to make these advances possible. However, there has been a growing awareness within the medical community and the patient population of the potential adverse effects of heart surgery and cardiopulmonary bypass surgery. Chief among these concerns is the potential of stroke or neurological deficit associated with heart surgery and cardiopulmonary bypass. One of the likely causes for stroke and neurological deficit is the release of emboli into the blood stream during heart surgery. Potential embolic material includes atherosclerotic plaque or calcified plaque from within the ascending aorta or cardiac valves and thrombus from within in the chambers of the heart. These potential emboli may be dislodged during surgical manipulation of the heart and/or the aorta, for example from cutting into the aortic wall to provide cannula access, or from the use of a cross clamp to block the connection of the aorta to the heart. In addition, the high velocity jetting effect from an aortic perfusion cannula may also cause dislodging of emboli. Emboli that flow to the brain may cause a stroke or neurological deficit. Clinical studies have shown a correlation between the number and size of emboli passing through carotid arteries and frequency and severity of neurological damage. Strokes have been associated with macroemboli larger than approximately 150 micrometers whereas emboli of approximately 150 micrometers or smaller cause more subtle neurological deficits.

Several medical articles have been published relating to cerebral embolic events and adverse cerebral outcomes associated with bypass surgery, including Roach et al, New England Journal of Medicine, vol. 335 No 25 1996 pp. 1857-1863; and Barbut et al, Stroke vol. 25 pp. 2398-2402, 1994.

There are many concepts and deployment mechanisms for preventing emboli from entering the coronaries and carotid arteries. One concept involves “filtering” the emboli from blood flow in the vessel. Currently, there are three basic concepts disclosed for an aortic filter, i.e., a cone shape, a parachute shape, and a “fish net” structure. Typically, the fish net is attached to a simple hoop, and the hoop enters the artery in a perpendicular fashion, i.e. edgewise. The cone is typically attached to a wire frame, while the parachute may or may not have a wire frame and is tethered to the deployment shaft.

There is also passive and active deployment. In passive deployment, the filter mouth is preloaded to spontaneously open, customarily using a sleeve to keep it closed or collapsed. When the sleeve is pulled back, the spring loaded wire frame opens. Another form of passive deployment requires that blood flow and subsequent pressure drop across the filter will force the filter open, similar to the action of a parachute. Examples of the active deployment system are an annular shaped balloon attached to the mouth of the filter, and a hollow hoop with a stiffening rod to open the hoop.

These designs have some disadvantages. First, there is a possibility that they may not form a tight seal against the aortic wall due to blood flow following the path of least resistance and flowing around the filter, between the filter and the arterial wall, thus possibly allowing emboli to escape downstream. Another disadvantage is that the deployment mechanism and/or the filter itself once positioned (for example perpendicular to the aorta) may occupy the same space as the heart pump cannula, thus causing the two to bump into each other, or to be incorrectly oriented. In addition, the filter itself may not be capable of allowing sufficient blood to pass through to carry enough oxygen to vital organs such as the brain, lungs, heart, kidneys, etc.

The fish net design is a commercially available device for filtering emboli during bypass surgery. As described in Maahs (U.S. Pat. No. 5,846,260), this filter is attached to the heart-lung machine cannula. The filter is configured such that the filter is located upstream from the cannula outlet, i.e., between the aortic clamp and the cannula. This provides no embolic protection caused for the output of the cannula and no protection during placement of the cannula. This filter provides protection only during manipulation of the clamp.

Tsugita (U.S. Pat. No. 5,910,154) discloses various filter designs. The basic premise of these designs is a passive method of a spring-loaded open wire frame with a filter attached, mounted on a guide wire. The filters are deployed in either a cone or a parachute configuration. The wire frame is collapsed or constrained using a sheath, which slides over the filter to collapse it for insertion or removal. The wire frame opens when a restraining sheath is pulled back.

Other concepts include introducing a shunt apparatus to isolate a segment of a patient's cardiovascular system and for directing circulatory flow around the isolated segment through a catheter shaft. The shunt apparatus, if introduced in the aortic arch, helps ensure that small or no amounts of emboli flow to the brain through the aortic arch vessels leading thereto.

### SUMMARY

The present disclosure relates to an apparatus and method for occluding the flow of embolic blood from entering the brain or other arteries during surgical procedures. The apparatus includes an expandable coiled occluder. The occluder is deployed in a coiled state into a body lumen where it then partially uncoils and at least partially conforms to the diameter of the lumen. The method includes introducing an occluder into a body lumen; having the occluder in a deployed/uncoiled position when emboli are likely to be released; and, if deemed necessary or desirable, having the occluder removed or coiled when emboli are not likely to be released.

In a particularly useful embodiment, the occluder is configured as an aortic shunt apparatus for deployment within a patient's aortic arch and methods are described for isolating the aortic arch vessels from the aortic lumen, for selectively perfusing the arch vessels with a fluid and for directing blood flow within the aortic lumen through an occluder past the isolated arch vessels. The occluder may be inserted into the aortic arch through the femoral artery via a catheter or cannula. The occluder may be used in combination with a bypass diverter, which supplies blood to the body during the surgical procedure, with the bypass diverter inserted into the arterial arch from a location upstream of the arterial vessels. Preferably, the occluder is inserted prior to placement of the bypass cannula. The occluder protects the patient from cerebral embolization and embolic stroke during cardiopulmonary bypass or cardiac surgery by directing potential emboli downstream from the aortic arch vessels where they will be better tolerated by the body. The occluder further protects the patient from cerebral hypoperfusion by providing selective perfusion of the aortic arch vessels and the cerebrovascular circulation with oxygenated blood or with neuroprotective fluids. The occluder also finds application for selective perfusion of the cerebrovascular circulation in the presence of risk factors, such as head trauma or cardiac insufficiency. The occluder will also find application for selective perfusion of other organ systems within the body.

In several embodiments, the occluder includes an expandable coiled sheet, which at least partially expands to conform to the shape of a body lumen. The occluder is inserted through the femoral artery in a contracted, coiled state. Once the occluder is in place, it uncoils and thus expands to conform to the diameter of the lumen.

It is envisioned for a set of tethers to be attached to both ends of the occluder and for a telescoping shaft to connect to each set of tethers and to extend through the occluder. In this embodiment, extending the telescoping shaft would interact with the tethers to collapse the occluder. The collapsed occluder could then fit through a deployment catheter or a delivery sheath. Similarly, shortening the telescoping shaft would, together with the sets of tethers, would allow the occluder to expand.

In one embodiment, only the leading (upstream) portion of the occluder is expandable and the remainder of the occluder is a hollow, cylindrical-shaped tube which extends from the coiled portion. In this embodiment, the leading edge of the occluder expands and conforms to the shape of the lumen, while the trailing portion is supported in an open configuration under pressure of the flow of blood through the aorta. The trailing portion covers the junction between the aorta and the aortic arch vessels, thus blocking blood from entering the aortic arch vessels.

In another embodiment, both the leading and trailing (downstream) portions of the occluder are expandable, while the middle portion of the occluder is a hollow, cylindrical-shaped tube which connects the leading portion to the trailing portion. In this embodiment, the aortic arch vessels are actively blocked off by the occluder on both the upstream and downstream portions with the middle portion extending therebetween.

In another embodiment, the occluder is made of a braided, woven or knitted structure. In this embodiment, the occluder can be expanded and contracted/collapsed by altering the braid angle. Since such a braided structure would be able to naturally expand and contract, thus a coiled portion would not be necessary. Additionally, a coating (e.g., silicone) can be applied to the braided structure to allow for complete occlusion.

In another embodiment, the occluder includes a plurality of holes spaced around its perimeter. This embodiment allows some of the blood entering the occluder to exit the occluder through these holes and travel to the aortic arch vessels, thus providing the brain with blood, which possibly contains emboli. In a particularly useful embodiment, the holes have a diameter of less than 150 micrometers. In this embodiment, embolic particles that are 150 micrometers or larger are not able to pass through the holes of the occluder as it is known in the art that embolic particles greater than 150 micrometers are the most harmful type of emboli.

It is also envisioned that the occluder may be made from a mesh material. This mesh tube would allow a relatively large amount of blood to pass from the occluder through the aortic arch vessels, while still filtering some of the emboli in the blood.

It is envisioned that instead of the coiled, expandable portions, the leading and/or trailing portions of the occluder can include sealing members that are inflatable toroidal balloon cuffs which can expand to create a seal between the shunt apparatus and the inner wall of the aortic lumen, thus confining the flow of embolic blood from entering the aortic arch vessels.

The method of the disclosure includes inserting the occluder into a body lumen and uncoiling/inflating at least a portion of the occluder to restrict the flow of blood through side or branch vessels. The method further includes determining whether the occluder should remain in place or whether it should be removed/coiled/deflated during portions of a surgical procedure to allow blood to flow to the restricted organ. If it is determined that the organ is not receiving enough blood during the surgical procedure, the occluder is then removed/coiled/deflated, allowing blood to reach the previously occluded organ. To allow blood to flow to the organ and to best protect the patient from embolic blood reaching the brain, for example, the occluder would be re-activated at certain key times during a surgical procedure when emboli are most likely to be dislodged.

### BRIEF DESCRIPTION OF THE DRAWINGS

Various embodiments of the subject instrument are described herein with reference to the drawings wherein:

FIG. 1 is a front view of an occluder apparatus according to an aspect of the present disclosure, the occluder apparatus being a tube having a coiled sheet along its entire length and is shown in an aortic arch with a set of tethers on each end and a telescoping shaft connecting the sets of tethers, a catheter shaft being positioned in the aortic arch on the upstream side of the occluder apparatus;

FIG. 2 is a cross-sectional view of the occluder apparatus taken along line 2-2 of FIG. 1;

FIG. 3 is a front view of the occluder apparatus of FIG. 1, the occluder apparatus being passively expandable along most of its length and having an expandable sealing member at its leading edge;

FIG. 4 is a front view of the occluder apparatus of FIG. 1, the occluder apparatus being passively expandable along most of its length and having an expandable sealing member at its leading edge and at its trailing edge;

FIG. 5 is a front view of the occluder apparatus of FIG. 1 illustrating an embodiment of an occluder apparatus having a plurality of holes disposed therethrough;

FIG. 6 is a front view of the occluder apparatus of FIG. 1 illustrating an embodiment of an occluder apparatus made of a mesh or filter material;

FIG. 7 is a front view of the occluder apparatus of FIG. 1 illustrating balloon-type sealing members at the leading edge and trailing edge of the occluder apparatus; and

FIG. 8 is a front view of an occluder apparatus having a braided structure along its length.

### DETAILED DESCRIPTION

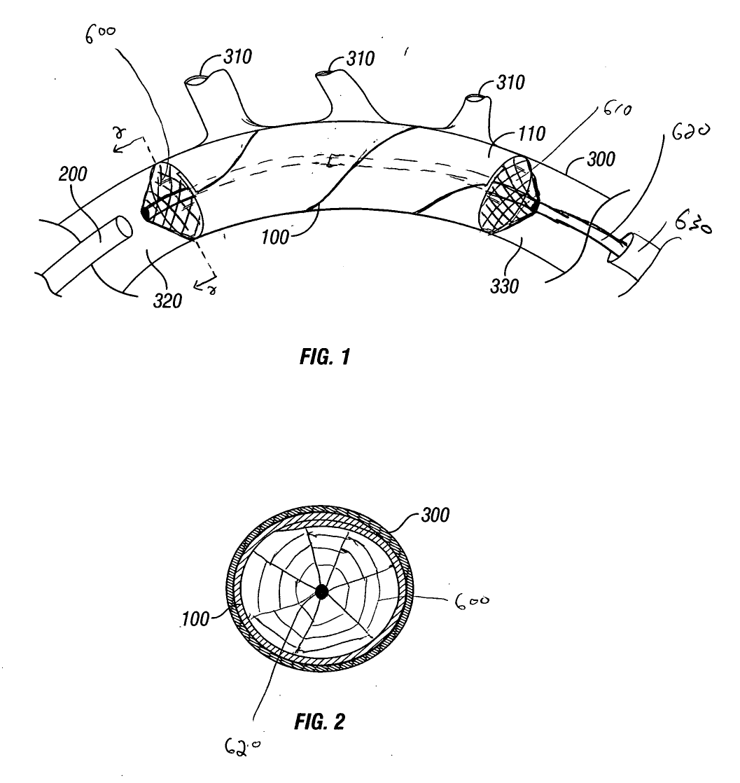

FIGS. 1-8 illustrate an occluder apparatus 100 according to an apparatus and method of the present disclosure. While the figures depict the occluder apparatus 100 positioned in an aortic arch 300, the present disclosure may be used in other suitable bodily locations to occlude or filter blood flow to side or branch vessels. For the purposes herein, the occluder apparatus 100 is described in terms of being used during a Coronary Artery Bypass Graft (CABG), however, it is contemplated that the occluder apparatus 100 can also be used during other open heart procedures, such as open valve replacements, aortic aneurysm repair, heart transplants, etc., and during endovascular procedures, such as coronary angioplasty/stenting, cardiac catheterization, etc. It is also contemplated that the occluder may find application, for example, to occlude the renal arteries during some surgical procedures.

The occluder apparatus 100 of FIG. 1 is an expandable coiled tube 110. It is envisioned that occluder apparatus 100 is made of a synthetic material (such as, e.g. a sheet, weave or knit of expanded PTFE, polypropylene, polyethylene, or polyurethane). Expandable coiled tube 110 is shown positioned in an aortic arch 300. Expandable coiled tube 110 spans from an upstream portion 320 (ascending aorta) to a downstream portion 330 (descending aorta) of aortic arch 300. A bypass catheter 200 is shown inserted into an upstream portion 320 of aortic arch 300. Bypass catheter 200 supplies the body with blood during the surgical procedure. An upstream set of tethers 600 and a downstream set of tethers 610 are also shown connected to a telescoping shaft 620. Center portion of telescoping shaft 620 that runs through occluder apparatus 100 is shown in phantom in FIG. 1 and is omitted in the other Figures for clarity. The spacing between individual tethers 600, 610 is preferably in the range from about 1000 micrometers to about 2000 micrometers and tethers 600, 610 are not designed to filter blood (the spacing is shown larger in the Figures for clarity).

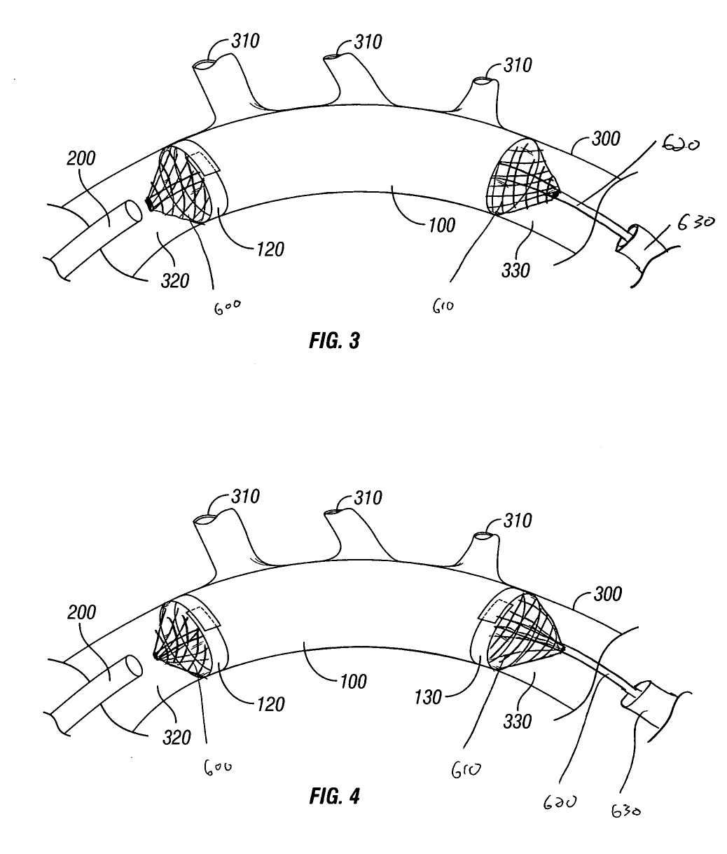

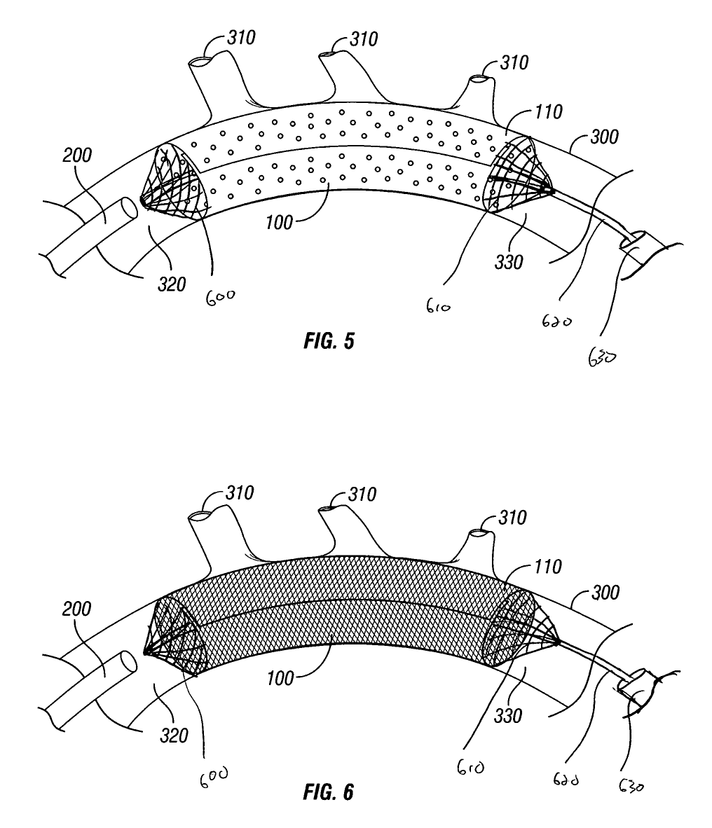

The occluder apparatus 100 can be inserted into the aortic arch 300 through the femoral artery (not shown), from the downstream portion 330 of the aortic arch 300. Occluder apparatus 100 generally has a reduced diameter for delivery which can be achieved by manipulation of telescoping shaft 620. Occluder apparatus 100 can be contracted to the reduced diameter by winding the expandable coiled tube 110 in a series of overlapping turns and/or by extending telescoping shaft 620. The expandable coiled tube 110 can be coiled in a spiral fashion (e.g., FIG. 1) or in a linear fashion (e.g., FIG. 5). Contracted occluder apparatus 100 can be accepted by a delivery sheath or similar device 630 for delivery to the target bodily location. Once the target location (e.g., aortic arch 300) is reached, the occluder apparatus 100 is released from the delivery sheath 630 and at least partially uncoils to substantially conform to the diameter of the aortic arch 300. Moreover, telescoping shaft 620 can be shortened to compress sets of tethers 600, 610 for allowing occluder apparatus 100 to at least partially uncoil. When in place, expandable coiled tube 110 partially uncoils to substantially conform to an inner wall of the artic arch 300, thus blocking off the aortic arch vessels 310 and preventing embolic blood from entering the aortic arch vessels 310. In other embodiments of the present disclosure (discussed below and illustrated in FIGS. 3, 4 and 7), only a portion of the occluder apparatus 100 includes an expandable/inflatable portion. As can be appreciated, a similar method of insertion can be utilized for such embodiments.

Removal of occluder apparatus 100 can be similarly be accomplished. To remove occluder apparatus 100 from aortic arch 300, expandable coiled portions 110, 120 and/or 130 (discussed below) or balloon portions 500 and/or 510 (discussed below) can be uncoiled or deflated, respectively, and pulled out through the downstream portion 330 of the aortic arch 300. Additionally, telescoping shaft 620 can be extended, thus stretching the sets of tethers 600, 610 and allowing occluder apparatus 100 to compress. As shown in FIGS. 3-8, telescoping shaft 620 similarly interacts with sets of tethers 600, 610 and occluder apparatus 100. It is particularly useful for downstream set of tethers 610 to be cone-shaped (as shown in FIGS. 1 and 3-8), while upstream set of tethers 600 can be either a cone-shaped set of tethers (FIGS. 1 and 3-7), a straight line set of tethers (FIG. 8) or any other reasonable configuration.

FIG. 2 illustrates a cross-section of expandable coiled tube 110 within a lumen, such as aortic arch 300. This figure shows expandable coiled tube 110 in an expanded state and snugly fits the inner diameter of aortic arch 300. Such a tight fit prevents blood from entering aortic arch vessels 310. The upstream tether set 600 is also shown. Individual tethers of tether set 600 connect to telescoping shaft 620 in the center and to expandable coiled tube 110 around its perimeter.

FIG. 3 illustrates an embodiment of occluder apparatus 100 having an upstream coiled sealing member 120 at its leading end. The remainder of occluder apparatus 100, in this embodiment, extends from upstream coiled sealing member 120 past the arch vessels 310 and is not actively expanded, but rather passively expands under pressure of the blood flowing through the aorta. In this embodiment, upstream coiled sealing member 120 expands to create a seal at a location upstream 320 of aortic arch vessels 310, and the middle and trailing portions of the occluder expand under blood fluid pressure to cover the aortic arch vessels, thus preventing blood from entering aortic arch vessels 310 from a location upstream 320 of occluder apparatus 100. It is not likely that any blood will enter aortic arch vessels 310 in this embodiment because blood flows from the upstream portion 320 of aortic arch 300 to the downstream portion 330 of aortic arch 300, and thus the blood passes the location of aortic arch vessels 310 prior to exiting occluder apparatus 100.

FIG. 4 illustrates an embodiment of occluder apparatus 100 having an upstream coiled sealing member 120 at its leading end and a downstream coiled sealing member 130 at its trailing end. The remainder of occluder apparatus 100, in this embodiment, extends from upstream coiled sealing member 120 past arch vessels 310 to downstream coiled sealing member 130 and ispassively expanded under blood fluid pressure. In this embodiment, upstream and downstream coiled sealing members 120, 130 expand to create seals at a location upstream 320 and downstream 330 of aortic arch vessels 310, respectively, thus preventing embolic blood from reaching aortic arch vessels 310 from both locations upstream 320 and downstream 330 of occluder apparatus 100.

In another embodiment, illustrated in FIG. 5, the occluder apparatus 100 has a plurality of holes 400 disposed therethrough. These holes 400 allow some blood to pass from occluder apparatus 100 to aortic arch vessels 310, thus providing the patient's brain with oxygenated blood. In a particularly useful embodiment, the diameter of the holes 400 is less than 150 micrometers. This sizing is of particular importance because embolic particles that are larger than 150 micrometers in diameter are known to cause the most damage to the brain. These harmful emboli will not be able to pass through holes 400 of occluder apparatus 100 and will therefore not be able to reach the brain.

In another embodiment, illustrated in FIG. 6, the occluder apparatus 100 is made from a flow through filter material 410. This filter material 410 would allow a relatively large amount of blood to pass from occluder apparatus 100 to aortic arch vessels 310, while still filtering some of the emboli in the blood. In one embodiment, filter material 410 can be made of a perforated sheet of expanded PTFE, polypropylene, polyethylene, or polyurethane, or may be a woven or knitted structure made from any of the foregoing.

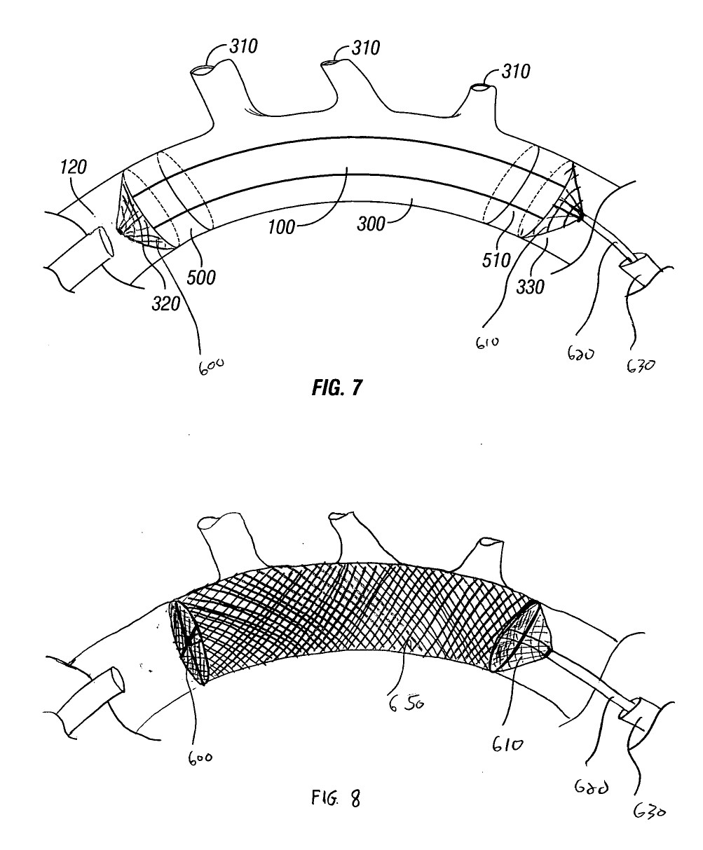

In another embodiment illustrated in FIG. 7, occluder apparatus 100 includes upstream balloon sealing member 500 and downstream balloon sealing member 510. These balloon sealing members 500, 510 are deflated during insertion of occluder apparatus 100 into aortic arch 300 and are inflated once occluder apparatus 100 is positioned in aortic arch 300. Similar to coiled sealing members 120, 130, balloon sealing members 500, 510 expand to create a seal at location upstream 320 and downstream 330 of aortic arch vessels 310, thus preventing blood from entering aortic arch vessels 310. It is also envisioned to only have an upstream balloon sealing member 500, and not a downstream balloon sealing member 510 (not illustrated). It is also envisioned for occluder apparatus 100 of this embodiment to include a plurality of holes 400 (as illustrated in and discussed with regard to FIG. 5, above) or for the occluder apparatus 100 to be made from a flow through filter material 410 (as illustrated in and discussed with regard to FIG. 6, above).

In another embodiment, illustrated in FIG. 8, the occluder apparatus 100 is made from a braided, woven or knitted material and forms a braided structure 650. Such a braided structure 650 is able to naturally expand and contract, without the need for a seam, by altering the braid angle. If complete occlusion is desired utilizing braided structure 650, a coating (e.g., silicone) can be used to coat the braided structure 650. Utilization of such a coating would still maintain the collapsible quality of the braided structure 650.

An important common feature of all of the foregoing embodiments is that the main body of the aorta remains unobstructed and blood is permitted to flow freely therethrough while the occluder is deployed. Prior filter devices which have attempted to filter full flow of the aorta have encountered difficulty due to creation of an unacceptable pressure gradient across filter medium capable of filtering embolic materials. In contrast, the filter of the present invention filters or occludes only flow to the aortic arch side or branch vessels, while permitting substantially unobstructed flow through the aorta to distal portions of the body.

In addition, it is contemplated that the filter may be coated with one or more anti-thrombolytic coatings, e.g. heparin or hyaluronic acid, or one or more lubricant or hydrolytic coatings to improve handling, e.g. during insertion and deployment and recovery within a femoral catheter.

Occluder apparatus 100 is used during surgical procedures to occlude embolic blood from reaching the aortic arch vessels 310 and the brain. Introduction of occluder apparatus 100 and catheter shaft 200 may be accomplished in accordance with known techniques for introducing catheters and related devices into the aortic arch through the femoral artery. Such techniques are commonly known in the art as described, for example, in U.S. Pat. No. 6,695,864 entitled “METHOD AND APPARATUS FOR CEREBRAL EMBOLIC PROTECTION” by Macoviak, et al., which is hereby incorporated by reference in its entirety herein.

A method of this disclosure involves introducing occluder apparatus 100 and catheter shaft 200 into aortic arch 300; deploying the occluder apparatus 100 (e.g., manipulating telescoping shaft 620, uncoiling expandable coiled tube 110 or uncoiling/inflating the sealing members 120, 130, 500 and/or 510) prior to and during steps of a surgical procedure when emboli are likely to be dislodged (e.g., cross-clamping the aorta, releasing cross clamps, etc); and taking down the occluder apparatus 100 (e.g., manipulating telescoping shaft 620, coiling/deflating sealing members 120, 130, 500 and/or 510 and/or moving occluder apparatus 100 away from aortic arch vessels 310, etc.) to perform surgical steps when emboli are not likely to be dislodged (e.g., during a CABG procedure) while allowing blood to flow into the aortic arch vessels. This method provides the brain with more non-embolic blood than it would receive using other methods.

More specifically, a method of the disclosure is described as follows, with respect to performing a CABG and inserting the occluder apparatus 100 into the aortic arch 300:
1) introduce and deploy occluder apparatus 100 into aortic arch 300 such that occluder apparatus 100 occludes flow to aortic arch vessels 310;
2) insert bypass catheter shaft 200 into aortic arch 300;
3) establish the flow of oxygenated blood to aortic arch 300 via catheter shaft 200;
4) cross-clamp the aorta (not shown) (stops the patient's heart);
5) deflate, coil and/or remove occluder apparatus 100;
6) perform CABG procedure;
7) redeploy, uncoil and/or inflate occluder apparatus 100;
8) release the cross clamp from the aorta (restarts patient's heart beat);
9) remove catheter shaft 200;
10) repair entry site of catheter shaft 200; and
11) remove occluder apparatus 100.

Another method of the present disclosure includes introducing an occluder apparatus 100 having a plurality of holes 400 (FIG. 5) or an occluder apparatus 100 being comprised of a flow through filter material 410 (FIG. 6) and the catheter shaft 200 into aortic arch 300; deploying occluder apparatus 100; determining if the blood flow through aortic arch vessels 310 is sufficient to support the brain during surgery; if blood flow is sufficient, having the occluder apparatus 100 remain in the aortic arch 300 throughout all or most of the surgery; if blood flow is not sufficient, taking down the occluder apparatus 100 during stages of the surgical procedure when emboli are not likely to be dislodged. To determine if blood flow through the aortic arch vessels 310 is sufficient, brain activity is monitored to determine whether the brain is under stress due to reduced blood flow to the brain through the aortic arch vessels, and a judgment is made by the surgeon whether the patient can tolerate having surgery performed with the occluder deployed during the procedure.

More specifically, this method of the disclosure is described as follows, with respect to performing a CABG and inserting occluder apparatus 100 comprised of a flow through filter material 410 or having a plurality of holes 400 into aortic arch 300:
1) introduce and deploy occluder apparatus 100 comprised of a flow through filter material 410 or having a plurality of holes 400 into aortic arch 300 such that occluder apparatus 100 occludes blood from flowing to aortic arch vessels 310;
2) insert catheter shaft 200 into aortic arch 300;
3) establish the flow of oxygenated blood to aortic arch 300 via catheter shaft 200;
4) cross-clamp the aorta (not shown) (stops the patient's heart);
5) determine if aortic vessels 310 are receiving enough blood;
   a. if aortic vessels 310 are receiving enough blood, leave occluder apparatus 100 in place; perform CABG procedure
   b. if aortic vessels 310 are not receiving enough blood, deflate, coil, deflate and/or remove occluder apparatus 100; perform CABG procedure; redeploy, uncoil and/or inflate occluder apparatus 100;
6) release the cross clamp from the aorta (restarts patient's heart beat);
7) remove catheter shaft 200;
8) repair the entry site of catheter shaft 200; and
9) remove the occluder apparatus 100.

While several embodiments of the disclosure have been shown in the figures, it is not intended that the disclosure be limited thereto, as it is intended that the disclosure be as broad in scope as the art will allow and that the specification be read likewise. Therefore, the above description should not be construed as limiting, but merely as exemplifications of preferred embodiments. Those skilled in the art will envision other modifications within the scope and spirit of the claims appended hereto.

What is claimed is:

1. A method of cerebral embolic protection including the steps of:
providing an occluder apparatus having an open center;
inserting the occluder apparatus into a patient;
positioning the occluder apparatus in a patient's aortic arch such that the occluder apparatus directs the flow of blood in the aorta past aortic arch to block blood from flowing to aortic arch vessels during times when embolic discharge is likely; and
positioning the occluder apparatus to allow blood to flow to aortic arch vessels during times when embolic discharge is not likely.

2. The method of claim 1 wherein the step of providing an occluder apparatus further includes providing an occluder apparatus being at least partially in the form of an expandable coiled tube.

3. The method of claim 2 wherein the expandable coiled tube forms a majority of the surface of the occluder apparatus.

4. The method of claim 2 wherein the expandable coiled tube being disposed adjacent a leading end of the occluder apparatus.

5. The method of claim 4 wherein the expandable coiled tube further including an expandable coiled tube portion being disposed adjacent a trailing end of the occluder apparatus.

6. The method of claim 1 wherein the step of providing an occluder apparatus further includes providing an occluder apparatus having an inflatable sealing member adjacent a leading end.

7. The method of claim 6 wherein the occluder apparatus further includes an inflatable sealing member adjacent a trailing end.

8. The method of claim 1 wherein the step of inserting the occluder apparatus into a patient is further defined by inserting the occluder apparatus through a patient's femoral artery.

9. An apparatus for use in surgical procedures to direct the flow of blood; the apparatus including
an occluder for insertion into an aortic arch, the occluder being at least partially in the form of an expandable coiled tube.

10. The apparatus of claim 9 wherein the expandable coiled tube forms a majority of the surface of the occluder.

11. The apparatus of claim 9 wherein the expandable coiled tube being disposed adjacent a leading end of the occluder.

12. The apparatus of claim 11, further including an expandable coiled tube portion being disposed adjacent a trailing end of the occluder.

13. The apparatus of claim 9 wherein the occluder includes a plurality of holes which allow blood to pass therethrough.

14. The apparatus of claim 15 wherein the maximum diameter of each of the plurality of holes is 150 micrometers.

15. The method of claim 9 wherein at least a portion of the occluder is comprised of a mesh filter material that allows blood to pass therethrough.

16. A method of cerebral embolic protection including the steps of:
providing an occluder apparatus having perforations configured and dimensioned to restrict passage of embolic matter;
inserting the occluder apparatus into a patient;
positioning the occluder apparatus in a patient's aortic arch in an activated position to limit the amount of blood from flowing to aortic arch vessels;
performing a cardiac surgery procedure on the patient; and
removing the occluder apparatus.

17. The method of claim 16 further comprising the steps of:
determining whether blood flow to the aortic arch vessels is sufficient to support the brain during a cardiac surgery procedure,
maintaining the occluder apparatus in the activated position throughout the cardiac surgery procedure if the blood flow to the aortic arch vessels is sufficient, positioning the occluder apparatus in an inactivated position during portions of the cardiac surgery procedure to allow more blood to flow to the aortic arch vessels if the blood flow to the aortic arch vessels is not sufficient to support the brain during the entire cardiac procedure.

18. The method of claim 16 wherein the step of providing an occluder apparatus is further defined by providing an occluder apparatus wherein the maximum diameter of each perforation is 150 micrometers.

19. The method of claim 16 wherein the step of providing an occluder apparatus is further defined by providing an occluder apparatus being comprised of a filter material.

20. The method of claim 16 wherein the step of providing an occluder apparatus is further defined by providing an occluder apparatus being at least partially in the form of an expandable coiled tube.

21. The method of claim 20 wherein the step of positioning the occluder apparatus in an activated position is further defined by uncoiling the expandable coiled tube.

22. The method of claim 20 wherein the step of removing the occluder apparatus is further defined by coiling the expandable coiled tube.

23. The method of claim 20 wherein the expandable coiled tube forms a majority of the surface of the occluder apparatus.

24. The method of claim 20 wherein the expandable coiled tube is disposed adjacent a leading end of the occluder apparatus.

25. The method of claim 20, further including an expandable coiled tube portion being disposed adjacent a trailing end of the occluder apparatus.

26. The method of claim 16 wherein the step of providing an occluder apparatus is further defined by providing an occluder apparatus having an inflatable sealing member adjacent a leading end of the occluder apparatus.

27. The method of claim 26 wherein the step of positioning the occluder apparatus in an activated position is further defined by inflating the inflatable sealing member.

28. The method of claim 26 wherein the step of removing the occluder apparatus is further defined by deflating the inflatable sealing member.

29. The method of claim 16 wherein the steps are performed during a coronary artery bypass grafting (CABG) procedure.
